(19) United States
(12) Patent Application Publication
(10) Pub. No.: US 2024/0041481 A1
(43) Pub. Date: Feb. 8, 2024

(54) DEVICES AND METHODS FOR CROSSING LESIONS IN A TISSUE LUMEN

(71) Applicant: Jihad Ali Mustapha, Ada, MI (US)

(72) Inventor: Jihad Ali Mustapha, Ada, MI (US)

(21) Appl. No.: 18/365,437

(22) Filed: Aug. 4, 2023

Related U.S. Application Data
(60) Provisional application No. 63/395,591, filed on Aug. 5, 2022.

(51) Int. Cl. A61B 17/22

(52) U.S. Cl. CPC A61B 17/22; A61B 2017/22094

(57) ABSTRACT

A device for crossing a lesion in a tissue lumen includes a crossing wire configured to pass through a lumen of a catheter, the crossing wire comprising a plurality of wire segments and the crossing wire configured to form a loop at a distal end of the crossing wire. The plurality of wire segments can include a plurality of wire links, with each wire link having a variable strength (or variable stiffness (e.g., flexibility). In another aspect, a device for crossing a lesion in a tissue lumen is provided having a predetermined pattern of variable strength from a proximal end to a distal end of the crossing wire.

### CROSS-REFERENCE TO RELATED APPLICATIONS

This application claims priority to U.S. Provisional Application No. 63/395,591 filed Aug. 5, 2022, the entire contents of which is hereby incorporated by reference.

### TECHNICAL FIELD

The present invention generally relates to crossing lesions in the vasculature, especially those referred to as chronic total occlusions (CTO) and/or high grade stenosis (HGS), and, more particularly, crossing devices and methods utilizing a loop feature.

### BACKGROUND

Vascular occlusions and, especially, CTOs and/or HGS can have a severe impact on a patient's health and lifestyle. There remains an unmet need for effective and reliable treatment options for crossing CTOs and/or HGS.

### BRIEF SUMMARY OF THE INVENTION

According to one embodiment, the present invention provides a device for crossing a lesion in a tissue lumen that includes a crossing wire configured to pass through a lumen of a catheter, the crossing wire comprising a plurality of wire segments and the crossing wire is configured to form a loop at a distal end of the crossing wire.

According to an embodiment, the crossing wire includes the loop.

According to an embodiment, the loop comprises at least two wire segments with a link between the at least two wire segments. According to one embodiment, the link has a variable gram force based on a wire diameter of the link. According to another embodiment, the link has at least three variations in gram force based on the wire diameter of the link. According to an embodiment, the link generates a gram force from a combination of the at least two wire segments that the link is positioned between.

According to an embodiment, the link comes into contact with the lesion.

According to an embodiment, the plurality of wire segments includes a plurality of wire links, with each wire link having a variable strength (or variable stiffness (e.g., flexibility)). According to another embodiment, each wire link of the plurality of wire links that has a variable strength alternates with a wire segment having a constant strength (or stiffness).

According to an embodiment, a wire link of the plurality of wire links is positioned at the distal end of the crossing wire. According to another embodiment, the wire link positioned at the distal end of the crossing wire has the lowest tensile strength. According to one embodiment, the wire link positioned at the distal end of the crossing wire has a tensile strength of less than 1 gram.

According to an embodiment, a wire link of the plurality of wire links is positioned at a proximal end of the crossing wire. According to another embodiment, the wire link positioned at the proximal end of the crossing wire has the highest tensile strength. According to one embodiment, the wire link positioned at the proximal end of the crossing wire has a tensile strength of greater than 200 grams.

According to an embodiment, each wire link of the plurality of wire links increases in stiffness (or strength (i.e., gram force)) from the distal end to a proximal end of the crossing wire. According to another embodiment, each wire link of the plurality of wire links has a strength of less than 1 gram to about 200 grams.

According to an embodiment, each wire link of the plurality of wire links increases in diameter from the distal end to a proximal end of the crossing wire. According to one embodiment, the diameter of each wire link of the plurality of wire links is one of (i) 0.014 inches, (ii) 0.018 inches, or (iii) 0.035 inches.

According to an embodiment, the plurality of wire segments comprises at least (i) a first segment at a proximal end of the crossing wire having a stiffness that is the highest stiffness of the crossing wire and (ii) a shaft attached to the first segment. According to one embodiment, the plurality of wire segments further comprises one or more of (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees. According to another embodiment, the plurality of wire segments further comprises a second segment at the distal end of the crossing wire having a stiffness that is at least one of (i) less than the stiffness of the first segment, or (ii) the lowest stiffness of the crossing wire.

According to an embodiment, the loop of the crossing wire is configured to be rotated (i) clockwise at 180 degrees and/or (ii) counterclockwise at 180 degrees. According to an embodiment, rotation of the loop narrows the size of the loop, which results in a loop that is straight with a smaller diameter and a higher strength.

According to an embodiment, the loop of the crossing wire is configured to be rotated (i) in a first direction and (ii) a second direction that is opposite to the first direction. According to one embodiment, rotation of the loop in (i) the first direction narrows the size of the loop, which results in a loop having a higher strength, and (ii) the second direction increases the size of the loop, which results in the loop having a lower strength.

According to an embodiment, the lesion comprises one or more of a chronic total occlusion (CTO) or a high grade stenosis (HGS).

According to an embodiment, a proximal end of the crossing wire has a first stiffness, and the loop has a second stiffness, wherein the first stiffness is greater than the second stiffness.

According to an embodiment, the crossing wire comprises at least two secondary wires that are twisted together to form the crossing wire. According to an embodiment, the crossing wire comprises at least three secondary wires that are twisted together to form the crossing wire.

According to an embodiment, the crossing wire is configured to be rotatable back and forth through an angle less than 360 degrees while maintaining contact with the lesion to erode the lesion. According to an embodiment, the crossing wire is configured to be rotatable back and forth through an angle of about 180 degrees while maintaining contact with the lesion to erode the lesion.

According to an embodiment, the crossing wire has a variable stiffness along its length. According to one embodiment, the loop includes a material that is radiopaque.

According to another embodiment, the present invention provides a method for crossing a chronic total occlusion (CTO) and/or a high grade stenosis (HGS) that includes inserting a catheter having a crossing wire disposed in a lumen of the catheter into an occluded vessel, the crossing wire comprising a plurality of wire segments and a loop at a distal end of the crossing wire; extending the loop of the crossing wire beyond a distal end of the catheter to contact an occlusion; grasping the crossing wire at a position proximal to a proximal end of the catheter; and rotating the grasped crossing wire back and forth through an angle less than 360 degrees while maintaining the loop of the crossing wire in contact with the occlusion to erode the occlusion.

According to one embodiment, the method further includes twisting the grasped crossing wire through an angle of about 180 degrees while pressing the loop of the crossing wire against the occlusion.

According to one embodiment, the plurality of wire segments comprises at least (i) a first segment at the proximal end of the crossing wire having a stiffness that is the highest stiffness of the crossing wire and (ii) a shaft attached to the first segment. According to an embodiment, the crossing wire is grasped at the first segment at the proximal end of the crossing wire and the first segment is rotated. According to another embodiment, the plurality of wire segments further comprises (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees. According to an embodiment, the step of twisting the grasped crossing wire causes the loop to form at one of the second portion, the third portion, or the fourth portion. According to an embodiment, the plurality of wire segments further comprises a second segment at the distal end of the crossing wire having a stiffness that is at least one of (i) less than the stiffness of the first segment, or (ii) the lowest stiffness of the crossing wire.

According to one embodiment, further comprising at least one of (a) rotating the grasped crossing wire in a first direction, or (b) rotating the grasped crossing wire in a second direction that is opposite to the first direction, wherein rotating the grasped crossing wire in the first direction narrows the size of the loop, which results in a loop having a higher strength, and wherein rotating the grasped crossing wire in the second direction increases the size of the loop, which results in the loop having a lower strength.

According to another embodiment, the present invention provides a device for crossing a lesion in a tissue lumen, the device comprising a crossing wire having a predetermined pattern of variable strength from a proximal end to a distal end of the crossing wire.

According to one embodiment, the crossing wire is configured to form a loop at the distal end of the crossing wire. According to another embodiment, the crossing wire further comprises a loop formed at the distal end of the crossing wire.

According to one embodiment, the crossing wire comprises a plurality of wire segments. In an embodiment, the plurality of wire segments includes a plurality of wire links, with each wire link having a variable strength. In an embodiment, each wire link of the plurality of wire links that has a variable strength alternates with a wire segment having a constant strength. In an embodiment, a wire link of the plurality of wire links is positioned at the distal end of the crossing wire. In an embodiment, the wire link positioned at the distal end of the crossing wire has the lowest tensile strength. In an embodiment, the wire link positioned at the distal end of the crossing wire has a tensile strength of less than 1 gram. In an embodiment, a wire link of the plurality of wire links is positioned at the proximal end of the crossing wire. In another embodiment, the wire link positioned at the proximal end of the crossing wire has the highest tensile strength. In an embodiment, the wire link positioned at the proximal end of the crossing wire has a tensile strength of greater than 200 grams. In an embodiment, each wire link of the plurality of wire links increases in stiffness (or strength) from the distal end to the proximal end of the crossing wire. In another embodiment, each wire link of the plurality of wire links has a strength of less than 1 gram to about 200 gram. In an embodiment, each wire link of the plurality of wire links increases in diameter from the distal end to a proximal end of the crossing wire. In an embodiment, the diameter of each wire link of the plurality of wire links is one of (i) 0.014 inches, (ii) 0.018 inches, or (iii) 0.035 inches.

In an embodiment, the plurality of wire segments comprises at least (i) a first segment at the proximal end of the crossing wire having a stiffness that is the highest stiffness of the crossing wire and (ii) a shaft attached to the first segment. In another embodiment, the plurality of wire segments further comprises one or more of (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees. In an embodiment, the plurality of wire segments further comprises a second segment at the distal end of the crossing wire having a stiffness that is at least one of (i) less than the stiffness of the first segment, or (ii) the lowest stiffness of the crossing wire.

In an embodiment, the lesion comprises one or more of a chronic total occlusion (CTO) or a high grade stenosis (HGS).

In an embodiment, the proximal end of the crossing wire has a first stiffness, and the loop has a second stiffness, wherein the first stiffness is greater than the second stiffness.

### BRIEF DESCRIPTION OF THE DRAWINGS

Further objectives and advantages will become apparent from a consideration of the description, drawings, and examples.

FIG. 1 illustrates a device for crossing a lesion that includes a crossing wire having a plurality of wire segments according to an embodiment of the invention

FIGS. 2A and 2B illustrate a device for crossing a lesion that includes a crossing wire in varying states of rotation (A to D) according to an embodiment of the invention.

FIG. 3 illustrates a device for crossing a lesion that includes a crossing wire in varying states of rotation (A to C) according to an embodiment of the invention.

FIG. 4 illustrates three examples of different crossing wires according to an embodiment of the invention.

FIGS. 5A and 5B illustrate a crossing wire prepared from three separate secondary wires according to an embodiment of the invention.

FIG. 6 illustrates a portion of a device for crossing a lesion according to an embodiment of the invention.

FIG. 7 illustrates a device inside a vessel lumen with a CTO according to an embodiment of the invention.

FIG. 8 illustrates the arteries below the knee as an example vascular in which the device can be used according to an embodiment of the invention.

FIGS. 9A to 9D illustrate a device for crossing a lesion that includes a crossing wire in varying states of rotation according to an embodiment of the invention.

FIGS. 10A and 10B illustrate a device for crossing a lesion that includes a crossing wire in varying states of rotation according to an embodiment of the invention.

### DETAILED DESCRIPTION OF THE INVENTION

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they may. Furthermore, the phrases “in another embodiment” and “in some other embodiments” as used herein do not necessarily refer to a different embodiment, although they may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising,” “including,” and “having” do not limit the scope of a specific claim to the materials or steps recited by the claim.

Vascular occlusions and, especially, CTOs and/or HGS can have a severe impact on a patient's health and lifestyle. CTOs and/or HGS are frequently encountered during endovascular interventions, with CTOs and HGS often being combined together. CTOs and/or HGS exist in many patients with symptomatic peripheral arterial disease. In the lower extremities, CTOs and/or HGS are commonly encountered in the superficial femoral artery (SFA). Crossing these lesions may be challenging and may lead to prolonged procedure time, increased operator and patient radiation exposure, high contrast load, and peri-procedural complications including perforation, dissection, loss of collaterals, and creation of an arteriovenous fistula.

Revascularization of CTOs and/or HGS is usually hindered by failure to cross the lesion due to a variety of factors, so attempts to revascularize heavily calcified CTOs and/or HGS still can meet with failure. Existing CTO and/or HGS crossing devices still have a higher failure rate than desirable. Further, existing devices are too large to use in the vasculature below the waist. There remains an unmet need for devices and methods that can reliably and effectively cross lesions.

There further remains a need for devices and methods that can be used below the waist, including in the legs, such as the legs of diabetic patients that experience particularly difficult blockages, including peripheral artery disease (PAD). Such devices and methods would allow physicians to reliably and effectively cross the CTO and/or HGS without consequences, such as, e.g., perforating the vessel wall while attempting to cross the CTO and/or HGS. There remains an unmet need for effective and reliable treatment options for crossing CTOs and/or HGS.

The present invention relates to devices and methods configured to reliably and effectively cross lesions in the vasculature, especially, lesions of the type where the accumulation of plaque is so severe that it results in a complete or nearly complete blockage of the vessel. The devices and methods in accordance with the principles of the invention are configured and adapted to cross an occlusion in order that interventional treatments can follow. The devices and methods described can include a crossing wire, a crossing catheter, and/or a combination of both utilized separately and/or in combination with each other and/or in combination with conventional wires and/or catheters.

The crossing devices and methods can include a guidewire or crossing wire having a distal end configured for more reliably crossing a chronic total occlusion (CTO) and/or a high grade stenosis (HGS). In one aspect, this configured distal end can be referred to as a loop, as discussed in more detail below. This loop-ended crossing wire is configured to present a more reliable device for interaction and engagement with the CTO and/or HGS, and, more particularly, a cap of the CTO and/or HGS that can have varying geometries and complexities. For example, CTO and/or HGS come in variable compositions, with some being severely calcified, some being moderately calcified, and some being mildly calcified. Moreover, HGS and CTO are often combined together. Thus, the loop-ended crossing wire is configured to present a more reliable device for advancing through an occlusion (e.g., CTO and/or HGS) to successfully cross the lesion. According to one embodiment, the looped-end crossing wire is able to create microfractures in the cap of the CTO and/or HGS. The cap of the CTO and/or HGS is the beginning of the CTO and/or HGS, which generally varies in thickness and/or content, which determines the resistance of the cap. For example, calcium, elastin, fibrin, and/or organized thrombus together determine the resistance of the CTO cap and/or the HGS cap. Thus, according to an embodiment, the looped-end crossing wire has pushability and/or the ability to be rotated right and/or left (i.e., clockwise and/or counterclockwise) by about 180 degrees, which accelerates the breakdown of the cap of the CTO and/or HGS. In addition, a narrowing of the loop or distal end of the crossing wire (due to, e.g., rotation of the crossing wire) allows for the looped-end crossing wire to enter an area with a larger diameter than an occlusion (e.g., a 100% occlusion), with the loop or distal end of the crossing wire being able to widen in size thereafter to the size of a new lumen.

The configuration at the end of the guide wire, referred to as a loop, can include various geometries, shapes, sizes and material properties and can be configured by way of the contemplated methods alone and/or in combination with guidewires and/or catheters. The loop feature, and the associated methods and/or devices, can be configured to present an interrogation conducive distal leading end that balances loop resiliency with stiffness so as to present itself optimally to the lesion yet also allow the loop to pass through the lesion. The loop configuration can be achieved by shape-memory and/or arrangements of wire segments alone, in combination with a catheter, and/or in combination with methods of use. For example, the guidewire loop can include a loop shape prior to use and/or a loop configuration that is formed in whole or in part in-situ, alone and/or in combination with a catheter.

A CTO crossing device according to some embodiments of the invention is directed to the concept of a loop at the distal end of the system, in particular, a guidewire loop. With this loop, the physician has the ability to use the leading distal end of the loop to interrogate the lesion and ultimately cross the lesion. The term “interrogate” as used herein can mean to contact, prod, probe, chip away at, break apart, dissect, and/or drill into a lesion. While interrogating a lesion may lead to crossing the lesion, the term “interrogating” is generally used to mean physically interacting with the lesion. The loop at the distal end of the system provides a stiffer surface for interrogating the lesion compared to, for example, using the floppy distal wire tip of a conventional guidewire.

Various aspects of the loop configuration can be considered. One aspect of the configuration is a dimensional configuration, such as the width of the loop. The width of the loop can be generally considered a lateral dimension. The width dimension of the loop can be configured based on the width or transverse dimension of the vessel in which the lesion is located. For example, the width may be configured to be half of the cross-sectional diameter of the vessel.

The loop can be configured to maintain geometries and/or configurations in use that allow crossing of the lesion without the loop collapsing and/or puncturing unintended areas of the vasculature. If the width of the loop exceeds the width of the vessel, or if the loop collapses, the vessel can rupture. The loop width can be selected to allow the loop to move along the vessel wall gently, without exerting point-like pressure on the vessel wall, and/or without exerting forces perpendicular to the vessel wall. According to one aspect, the width of the loop is between about 0.05 mm and about 6 mm. According to one aspect, the width of the loop is between about 1.5 mm and about 2.5 mm. According to one aspect, the width of the loop is between about 2.5 mm and about 6 mm.

In one aspect, the device can be configured to limit the width of the loop to be about half the width of the vessel. For example, for a 5 or 6 mm vessel, the width of the loop will less than about 2.5 or 3 mm. A narrow loop can move along the vessel wall without exerting point-like pressure on the vessel wall, and without exerting forces perpendicular to the vessel wall. If the width of the loop were allowed to expand such that it significantly exceeded the diameter of the vessel, the sides of the loop may exert forces perpendicular to the surface of the vessel wall that could puncture the vessel wall.

The loop can have a configuration that prevents a width of the loop from exceeding a width of the tissue lumen. The configuration may prevent the width of the loop from exceeding the width of the tissue lumen to the point of rupture, risk of rupture, undesirable stress and/or strain on the vessel, or beyond the vessel's elastic limits. In one aspect, the loop configuration may prevent the width of the loop from exceeding a width that is slightly greater than the diameter of the tissue lumen when no forces are being applied, because the shape of the lumen may change when an expanding force is applied by the loop, increasing the width of the lumen. In one aspect, the configuration may prevent the width of the loop from exceeding the width of the tissue lumen to the point of injury. In one aspect, the configuration may provide a loop that is not damaging to the healthy lumen size and/or shape of the lumen. In one aspect, the configuration controls the width of the loop to be about half the diameter of the tissue lumen or less. In one aspect, the configuration controls the width of the loop relative to the lumen diameter. In one aspect, the configuration controls the width of the loop relative to the lesion.

The distal-most portion of the loop is referred to herein as the leading distal end, or leading portion. The leading distal end of the loop can be configured in accordance with the principles of the invention to have a size and shape that are optimized for a particular application. For example, the leading distal end can be pointed, rounded, convex, or concave depending on the shape and hardness of the lesion to be interrogated.

The leading distal end of the loop can be configured to come into contact with the lesion. The distal end of the loop can be generally configured with a curvature of various types, some of which are shown in the drawings and discussed below. In one aspect, the leading distal end can be configured to be pointed, providing a smaller surface area for contacting the occlusion as compared to a loop having a rounded leading end. When the leading distal end contacts the lesion, the pointed leading distal end concentrates a force applied to the lesion over a smaller area of the lesion than a rounded leading distal end would.

The loop portion of the crossing wire can be pre-formed such that the leading distal end of the loop has a predetermined configuration. For example, the crossing wire can assume a looped or bent shape even when no external forces are acting on it. When no forces are acting on the loop, the configuration of the loop can be referred to as a “relaxed” or steady-state configuration. The fact that the loop is pre-formed helps maintain the narrow width of the loop, because the crossing wire itself will provide a counter-force when the loop is expanded beyond its pre-formed width. For example, when the leading distal end of the loop is brought into contact with a lesion and additional force is applied to the crossing wire, if the lesion resists the applied forces, the loop may begin to expand. However, the crossing wire itself will provide tensile forces that resist expansion of the loop beyond its preformed width. The widest portions of the loop can contact the vessel wall, and can stabilize the loop with respect to the vessel wall, such as the arterial wall.

In addition to the loop configuration contemplated in its basic form as discussed above in various aspects and configurations, the loop can further be configured to include more complex loop configurations. The additional loop configurations can have a single loop configuration as the basis of the loop configurations. The loop can be configured to allow for twisting and/or wrapping of the loop during use.

The loop alone and/or in combination with the catheter can be configured to be adaptable and/or controlled during use by methods and techniques contemplated herein. In one aspect, during use of the CTO crossing device, the operator, such as a physician, can control the crossing wire by displacement, for example, such as by rotation and/or twisting. For example, according to an embodiment, pushing the looped crossing wire forward in the vessel in combination with rotating the loop to the right and left (i.e., 180° rotation) allows for the loop to come into contact with the chronic total occlusion (e.g., CTO and/or HGS).

The present device enables the physician to control the width of the loop, thereby enhancing the safety and efficacy of the procedure. Some configurations of the invention also include a catheter, into which the looped crossing wire is disposed. By disposing the wire inside the catheter, the amount of bowing that the wire can undergo is limited. If the wire begins to bow inside the catheter, the catheter wall redirects the lateral forces so that they extend along the length of the catheter, and toward the leading distal end of the loop.

According to an embodiment of the invention, a multi-variation looped wire is provided that is designed with multiple wire links or segments that each represents a variable strength. According to an embodiment, a multi-variation looped wire or crossing wire is provided that has a predetermined pattern of variable strength from a proximal end to a distal end of the crossing wire. According to one embodiment, a distal end of the loop contains the lowest gram tensile strength, and the stiffness and/or gram tensile strength (or gram force) increase thereafter along a length of the wire. According to an embodiment, the increase in stiffness and/or gram tensile strength (or gram force) relates to a diameter of the wire. According to an embodiment, the multiple wire links or segments in combination with the wire diameter generates a variation in the gram force that is generated at each wire link and at the loop.

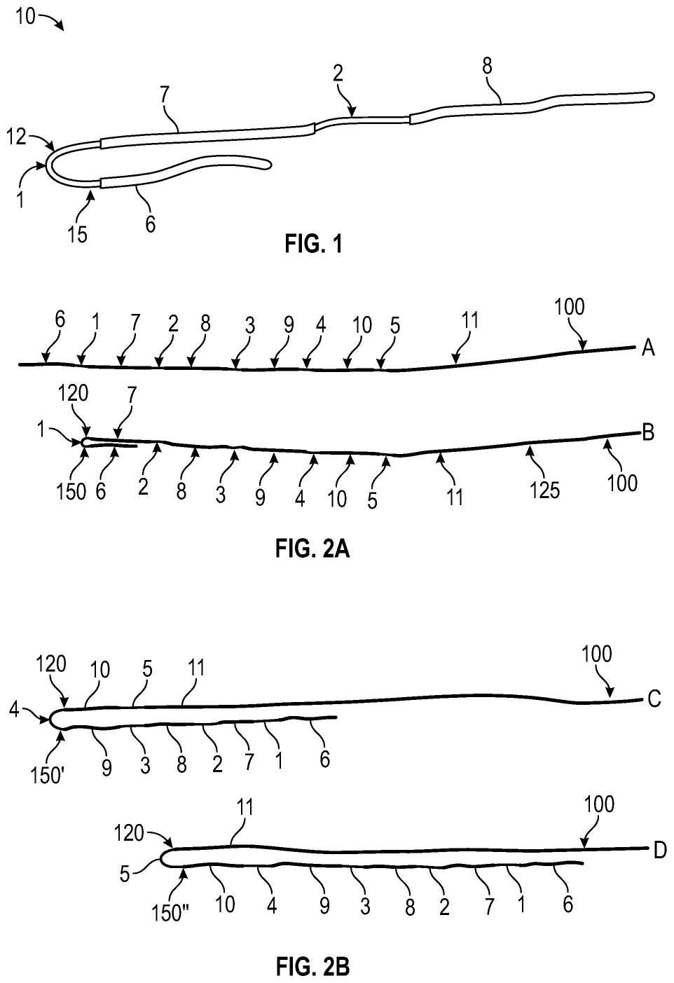

A device or wire for crossing a lesion that comprises a multi-variation looped wire according to some embodiments of the invention is shown in FIG. 1. As shown in FIG. 1, the looped wire 10 includes a plurality of wire segments (6, 7, 8) with a pair of links (1, 2) between respective wire segments (6, 7, 8). Link 1 is the most distal link and creates the loop 15 of the looped wire 10 at the distal end 12 of the looped wire 10. Link 1 has a variable tensile strength (or gram force) based on a diameter of the wire 10. According to one embodiment, the link 1 has three variations of tensile strength (or gram force) based on the diameter of the wire 10. In addition, the link 1 and the loop 15 created at this link 1 generates its tensile strength (or gram force) from a combination of the surrounding wire segments. For example, as shown in FIG. 1, the looped wire 10 has a loop 15 that is created by the link 1 and the two adjacent wire segments (6, 7) at the distal end 12 of the looped wire 10. As the looped wire 10 is pushed forward into a vessel, the loop 15, which includes the link 1 and the two adjacent wire segments (6, 7) at the distal end 12 of the looped wire 10, is the portion of the looped wire 10 that will come into contact with the chronic total occlusion (e.g., CTO and/or HGS). According to an embodiment, pushing the looped wire 10 forward in combination with rotating the tip (or the loop 15) to the right and left at 180° rotation, allows for the link 1, as well as the loop 15, to come into contact with the chronic total occlusion (e.g., CTO and/or HGS). In addition, the combination of the distal link 1 and the two adjacent wire segments (6, 7) has the ability to narrow the tip or loop 15 (e.g., by rotating the looped wire 10 to the right and left at 180° rotation) to accommodate an HGS, because as the loop 15 narrows, the loop 15 also becomes straight and smaller in diameter with a higher tensile strength. For example, according to an embodiment, the loop 15 of the looped wire 10 is positioned within a vessel using, e.g., a catheter, the loop 15 is first rotated to the right and left at 180°, such that the link 1 creates microfractures in a cap of the CTO or HGS. As discussed above, the cap is the beginning of the CTO or HGS, which varies in thickness and content, which determines the cap resistance. Calcium, elastin, fibrin, and/or organized thrombus together determine the cap resistance. According to an embodiment, the pushability of the looped wire 10 and the rotation to the right and left at 180° accelerates the breakdown of the cap. Hence, the narrowed wire tip or loop 15 enters an area with a larger diameter than a 100% occlusion (e.g., CTO or HGS). Thereafter, the loop 15 of the looped wire 10 can widen to the size of the new lumen (e.g., after breaking down the occlusion).

FIGS. 2A and 2B illustrate a multi-variation looped wire for crossing a lesion according to some embodiments of the invention. As shown in FIG. 2A, the looped wire 100 includes (i) a first configuration (A) in which a plurality of wire segments (6, 7, 8, 9, 10, 11) and a plurality of links (1, 2, 3, 4, 5) that alternate with the plurality of wire segments (6, 7, 8, 9, 10, 11) are disposed in a substantially straight line, and (ii) a second configuration (B) in which the plurality of wire segments (6, 7, 8, 9, 10, 11) and the plurality of links (1, 2, 3, 4, 5) that alternate with the plurality of wire segments (6, 7, 8, 9, 10, 11) are initially rotated to create a loop 150 at the distal end 120 of the looped wire 100. As shown in the second configuration (B) of FIG. 2A, the loop 150 is created with the link 1 and the two adjacent wire segments (6, 7). According to one embodiment, the first configuration (A) of FIG. 2A is the position in which the looped wire 100 enters the body. Thus, according to one embodiment, the looped wire 100 is within the first configuration (A) when the looped wire 100 enters the body, and is initially rotated into the second configuration (B) after the looped wire 100 is within the body. According to another embodiment, the second configuration (B) of FIG. 2A is the position in which the looped wire 100 enters the body. Thus, according to one embodiment, the looped wire 100 is put into the second configuration (B) prior to entering the body.

FIG. 2B illustrates the looped wire 100 in a third configuration (C) in which the plurality of wire segments (6, 7, 8, 9, 10, 11) and the plurality of links (1, 2, 3, 4, 5) that alternate with the plurality of wire segments (6, 7, 8, 9, 10, 11) are further rotated to create a loop 150′ at the distal end 120 of the looped wire 100. As shown in the third configuration (C) of FIG. 2B, the loop 150′ is created with the link 4 and the two adjacent wire segments (9, 10). FIG. 2B further illustrates the looped wire 100 in a fourth configuration (D) in which the plurality of wire segments (6, 7, 8, 9, 10, 11) and the plurality of links (1, 2, 3, 4, 5) that alternate with the plurality of wire segments (6, 7, 8, 9, 10, 11) are further rotated to create a loop 150″ at the distal end 120 of the looped wire 100. As shown in the fourth configuration (D) of FIG. 2B, the loop 150″ is created with the link 5 and the two adjacent wire segments (10, 11). According to one embodiment, the looped wire 100 enters the body either within the first configuration (A) of FIG. 2A or the second configuration (B) of FIG. 2A, and is thereafter rotated into the third configuration (C) of FIG. 2B and/or the fourth configuration (D) of FIG. 2B after being placed within the body.

According to one embodiment, the link (1, 2, 3, 4, 5) and its associated loop (e.g., 150, 150′, 150″) is created by rotating the looped wire 100 clockwise (i.e., 180° rotation), which thereby creates tension in the looped wire 100. Thus, as the looped wire 100 is rotated, a different link (1, 2, 3, 4, 5) and its associated loop (e.g., 150, 150′, 150″) is created that will be used to interrogate the lesion (CTO and/or HGS) and ultimately cross the lesion.

In the embodiment of the looped wire 100 in FIGS. 2A and 2B, the looped wire 100 includes (i) a first link 1 that is positioned between two adjacent wire segments (6, 7), (ii) a second link 2 that is positioned between two adjacent wire segments (7, 8), (iii) a third link 3 that is positioned between two adjacent wire segments (8, 9), (iv) a fourth link 4 that is positioned between two adjacent wire segments (9, 10), and (v) a fifth link 5 that is positioned between two adjacent wire segments (10, 11). According to one embodiment, each of the links (1, 2, 3, 4, 5) has a variable gram force based on a wire diameter of the respective link. According to another embodiment, each of the links (1, 2, 3, 4, 5) has at least three variations in gram force based on the wire diameter of the respective link. According to an embodiment, each of the links (1, 2, 3, 4, 5) generates a gram force from a combination of the at least two wire segments and the link that is positioned between.

According to an embodiment, the plurality of wire segments (6, 7, 8, 9, 10, 11) of FIGS. 2A and 2B alternate with the plurality of wire links (1, 2, 3, 4, 5), with each wire link (1, 2, 3, 4, 5) having a variable strength (or variable stiffness (e.g., flexibility)) and/or an increasing strength from the distal end 120 to a proximal end 125 of the looped wire 100. According to another embodiment, each wire link of the plurality of wire links (1, 2, 3, 4, 5) that has a variable strength alternates with a wire segment (6, 7, 8, 9, 10, 11) having a constant strength (or stiffness). According to one embodiment, the plurality of wire segments that alternate with plurality of wire links creates a crossing wire having a predetermined pattern of variable strength from a proximal end to a distal end of the crossing wire (as discussed further below).

According to an embodiment, the wire link 1 of the plurality of wire links (1, 2, 3, 4, 5) of FIG. 2A is positioned at the distal end 120 of the looped wire 100. According to one embodiment, the wire link 1 positioned at the distal end 120 of the looped wire 100 has the lowest tensile strength, such as, e.g., less than 1 gram. According to another embodiment, the wire link 5 of the plurality of wire links (1, 2, 3, 4, 5) of FIG. 2A is positioned at a proximal end 125 of the looped wire 100. According to one embodiment, the wire link 5 positioned at the proximal end 125 of the looped wire 100 has the highest tensile strength, such as, e.g., greater than 200 grams. According to an embodiment, each wire link (1, 2, 3, 4, 5) of the plurality of wire links (1, 2, 3, 4, 5) of FIGS. 2A and 2B increases in stiffness (or strength (i.e., gram force)) from the distal end 120 to the proximal end 125 of the looped wire 100. According to one embodiment, each wire link (1, 2, 3, 4, 5) of the plurality of wire links (1, 2, 3, 4, 5) of FIGS. 2A and 2B has a strength of less than 1 gram to about 200 grams. Thus, as the looped wire 100 is rotated clockwise (i.e., 180° rotation), the link (1, 2, 3, 4, 5) that creates the loop (e.g., loop 150, 150′, 150″) that will interrogate the lesion (CTO and/or HGS), and ultimately cross the lesion, will have a different stiffness (or strength (i.e., gram force)), based on the strength (i.e., gram force) of the specific link (1, 2, 3, 4, 5) and/or the tension created via rotating the looped wire 100 clockwise (i.e., 180° rotation).

According to one embodiment, about 400 grams of force is generated between the initial wire segment 6 of the looped wire 100 and the final wire segment 11 of the looped wire 100, based on the respective stiffness (or strength (i.e., gram force)) of each wire link (1, 2, 3, 4, 5) and each wire segment (6, 7, 8, 9, 10, 11) of the looped wire 100.

According to an embodiment, each wire link (1, 2, 3, 4, 5) of the plurality of wire links (1, 2, 3, 4, 5) of FIGS. 2A and 2B increases in diameter from the distal end 120 to the proximal end 125 of the looped wire 100. According to an embodiment, the range in stiffness (or strength (i.e., gram force)) of each wire link (1, 2, 3, 4, 5) of the plurality of wire links (1, 2, 3, 4, 5) of FIGS. 2A and 2B depends on the wire diameter at the respective wire link (1, 2, 3, 4, 5). For example, according to one embodiment, the wire diameter can range from 0.014 inches to 0.018 inches to 0.035 inches. The wire diameter of the respective wire link (1, 2, 3, 4, 5) in turn relates to the stiffness (or strength (i.e., gram force)) of the respective wire link (1, 2, 3, 4, 5) of the plurality of wire links (1, 2, 3, 4, 5) of FIGS. 2A and 2B, such that the respective wire link (1, 2, 3, 4, 5) can have a strength of less than 1 gram to about 200 grams depending upon its wire diameter. According to an embodiment, the combination of the wire link (1, 2, 3, 4, 5) of the plurality of wire links (1, 2, 3, 4, 5) of FIGS. 2A and 2B with their respective wire diameter generates the variation of the strength (i.e., gram force) at the respective wire link (1, 2, 3, 4, 5) and/or the loop of the looped wire 100 (see, e.g., loop 150 at link 1 in configuration (B) of FIG. 2A; loop 150′ at link 4 in configuration (C) in FIG. 2B; and loop 150″ at link 5 of configuration (D) of FIG. 2B).

According to an embodiment, the multi-variation looped wire can have a circular cross-section, with a certain wire diameter that, according to some embodiments, increases from the distal end to the proximal end of the looped wire. According to another embodiment, the multi-variation looped wire can have a rectangular cross-section, an oval shape, an oblong shape, a circular shape, or any combination thereof. These shapes are provided as examples, and the embodiments of the invention are not limited to these shapes.

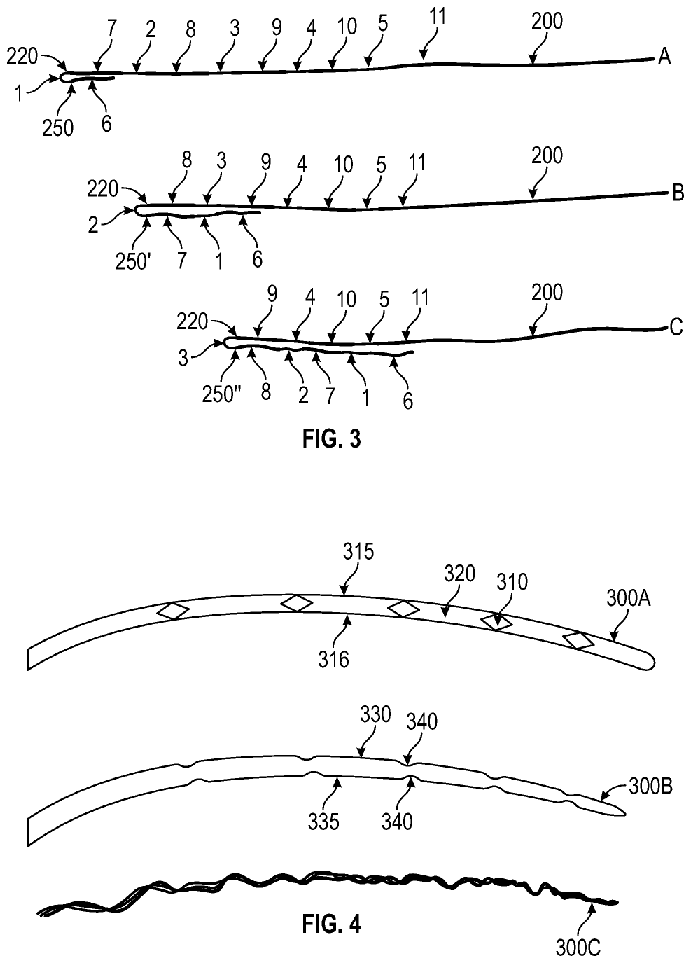

FIG. 3 illustrates a multi-variation looped wire for crossing a lesion according to some embodiments of the invention. As shown in FIG. 3, the looped wire 200 includes a first configuration (A) in which a plurality of wire segments (6, 7, 8, 9, 10, 11) and a plurality of links (1, 2, 3, 4, 5) that alternate with the plurality of wire segments (6, 7, 8, 9, 10, 11) are initially rotated to create a loop 250 at a distal end 220 of the looped wire 200. As shown in the first configuration (A) of FIG. 3, the loop 250 is created with the link 1 and the two adjacent wire segments (6, 7). FIG. 3 further illustrates the looped wire 200 in a second configuration (B) in which the plurality of wire segments (6, 7, 8, 9, 10, 11) and the plurality of links (1, 2, 3, 4, 5) that alternate with the plurality of wire segments (6, 7, 8, 9, 10, 11) are further rotated to create a loop 250′ at the distal end 220 of the looped wire 200. As shown in the second configuration (B) of FIG. 3, the loop 250′ is created with the link 2 and the two adjacent wire segments (7, 8). FIG. 3 also illustrates the looped wire 200 in a third configuration (C) in which the plurality of wire segments (6, 7, 8, 9, 10, 11) and the plurality of links (1, 2, 3, 4, 5) that alternate with the plurality of wire segments (6, 7, 8, 9, 10, 11) are further rotated to create a loop 250″ at the distal end 220 of the looped wire 200. As shown in the third configuration (C) of FIG. 3, the loop 250″ is created with the link 3 and the two adjacent wire segments (8, 9).

According to one embodiment, the link (1, 2, 3, 4, 5) and its associated loop (e.g., 250, 250′, 250″) is created by rotating the looped wire 200 clockwise (i.e., 180° rotation), which thereby creates tension in the looped wire 200. Thus, as the looped wire 200 is rotated, a different link (1, 2, 3, 4, 5) and its associated loop (e.g., 250, 250′, 250″) is created that will be used to interrogate the lesion (CTO and/or HGS) and ultimately cross the lesion.

In the embodiment of the looped wire 200 in FIG. 3, the looped wire 200 includes (i) a first link 1 that is positioned between two adjacent wire segments (6, 7), (ii) a second link 2 that is positioned between two adjacent wire segments (7, 8), (iii) a third link 3 that is positioned between two adjacent wire segments (8, 9), (iv) a fourth link 4 that is positioned between two adjacent wire segments (9, 10), and (v) a fifth link 5 that is positioned between two adjacent wire segments (10, 11). According to one embodiment, each of the links (1, 2, 3, 4, 5) has a variable gram force based on a wire diameter of the respective link. According to another embodiment, each of the links (1, 2, 3, 4, 5) has at least three variations in gram force based on the wire diameter of the respective link. According to an embodiment, each of the links (1, 2, 3, 4, 5) generates a gram force from a combination of the at least two wire segments that the link is positioned between.

According to an embodiment, the plurality of wire segments (6, 7, 8, 9, 10, 11) of FIG. 3 alternate with the plurality of wire links (1, 2, 3, 4, 5), with each wire link (1, 2, 3, 4, 5) having a variable strength (or variable stiffness (e.g., flexibility)). According to another embodiment, each wire link of the plurality of wire links (1, 2, 3, 4, 5) that has a variable strength alternates with a wire segment (6, 7, 8, 9, 10, 11) having a constant strength (or stiffness). Thus, as discussed above, according to an embodiment, the plurality of wire segments that alternate with plurality of wire links creates a crossing wire having a predetermined pattern of variable strength from a proximal end to a distal end of the crossing wire.

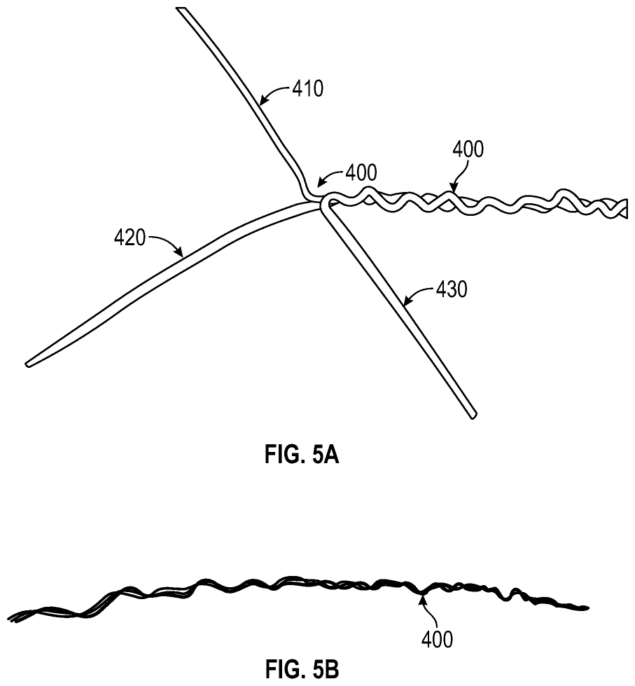

FIG. 4 illustrates three examples of different crossing wires according to an embodiment of the invention. As shown in FIG. 4, a first embodiment of a crossing wire 300A is illustrated that includes a first side 315, a second side 316 that is opposite to the first side 315, and a body region 320 therebetween. According to an embodiment, the body region 320 of the crossing wire 300A includes a plurality of openings 310 that provide flexibility to the body region 320 and the crossing wire 300A. FIG. 4 further illustrates a second embodiment of a crossing wire 300B that includes a first side 330 and a second side 335 that is opposite to the first side 330. According to this embodiment of the crossing wire 300B shown in FIG. 4, the first side 330 and the second side 335 include indented regions 340 that provide flexibility to the crossing wire 300B. FIG. 4 also illustrates a third embodiment of a crossing wire 300C. According to this embodiment, the crossing wire 300C is prepared from three independent wires, which will be further described below with respect to FIGS. 5A and 5B.

FIGS. 5A and 5B illustrate a crossing wire prepared from three separate secondary wires according to an embodiment of the invention (see also, e.g., crossing wire 300C of FIG. 4). As shown in FIG. 5A, a crossing wire 400 is created by weaving, wrapping and/or braiding three independent or separate secondary wires (410, 420, 430) together. For example, each of the separate secondary wires (410, 420, 430) are wrapped around each other or braided together to create the singular wire 400 shown in FIGS. 5A and 5B. According to one embodiment, each of the separate secondary wires (410, 420, 430) has a certain stiffness (or strength (i.e., gram force)). By wrapping or braiding these separate secondary wires (410, 420, 430) together, the prepared singular wire 400 will have an overall higher stiffness (or strength (i.e., gram force)), as compared to the stiffness (or strength (i.e., gram force)) of the separate secondary wires (410, 420, 430) alone.

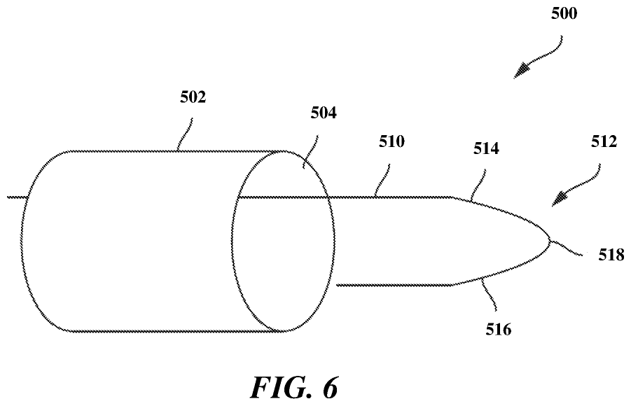

FIG. 6 illustrates a device for crossing a lesion (e.g., CTO and/or HGS) according to some embodiments of the invention. As shown in FIG. 6, the device 500 includes a catheter 502 including a lumen 504, the catheter 502 having a proximal end and a distal end. The device also includes a crossing wire 510 configured to pass through lumen 504, the crossing wire 510 including a loop 512 at a distal end of the crossing wire 510, the loop 512 having a relaxed state such that opposite sides 514, 516 of the loop 512 form an angle that is less than 180 degrees, and the loop 512 having a leading portion 518 configured to interrogate the lesion. The term “relaxed state” is intended to mean a state of the crossing wire when no external forces are exerted on it. For example, FIG. 6 shows a crossing wire 510 in a relaxed state. The opposite sides 514, 516 of the loop 512 can form an angle, with the angle being less than, e.g., 180 degrees. In one aspect of the invention, the angle is between about 90 and about degrees. In one aspect of the invention, the angle is between about 60 and about 30 degrees. The angle will influence the width of the looped portion of the crossing wire. A crossing wire with opposite sides that form an angle of 90 degrees in a relaxed state will form a wider loop than a crossing wire with opposite sides that form an angle of 45 degrees in a relaxed state. The angle may be chosen based on the diameter of the vessel, with smaller angles corresponding to smaller vessels and larger angles corresponding to larger vessels. Further, loops forming a wider angle may be chosen for navigating the true lumen of a vessel during a CTO crossing procedure, while loops forming a narrower angle may be chosen for navigating the subintimal region of the vessel, if the CTO cannot be crossed with the loop remaining in the true lumen.

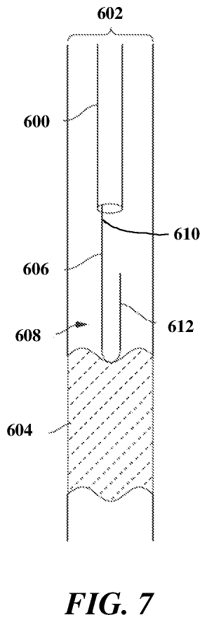

FIG. 7 shows a catheter 600 inside a vessel lumen 602. A CTO (and/or an HGS) 604 blocks the lumen 602. A crossing wire 606 is shown extending beyond the distal end of the catheter 600. The crossing wire 606 has a loop 608 that forms the distal end of the crossing wire 606. The loop 608 can come into contact with the CTO 604, and can be used by the physician to perform microdissection of the CTO 604, opening the vessel and creating a path for a guide wire or other device if further treatment is required. The physician my use multiple looped crossing or CTO wires to cross the CTO 604. For example, the physician may use a first looped crossing wire from an antegrade approach and a second looped crossing wire from a retrograde approach.

The crossing wire can undergo structural formation such that, when no forces are applied to the wire, the wire assumes the configuration or shape as shown, where a portion of the wire doubles back. For example, in FIG. 7, the crossing wire 606 has a main shaft 610, a loop 608, and a second shaft 612 that doubles back toward the catheter 600. The wire including the double-backed portion may form a V-shape, a U-shape, a W-shape, or an M-shape, for example. These shapes are provided as examples, and the embodiments of the invention are not limited to these shapes. The wire including the doubled-back portion may be referred to herein as “looped.” Loop 608 can be pre-formed, and can have shape memory characteristics. The shape memory characteristics allow the loop to resist forces that would cause the loop to become wider. For example, if the loop 608 is pre-formed to have a particular width, when a force is exerted on the loop that would cause the width of the loop to increase, tensile forces in the wire will resist the lateral forces, helping maintain the predetermined width of the loop. The loop 608 can be passable through the catheter and can assume its relaxed configuration in whole or in part for use.

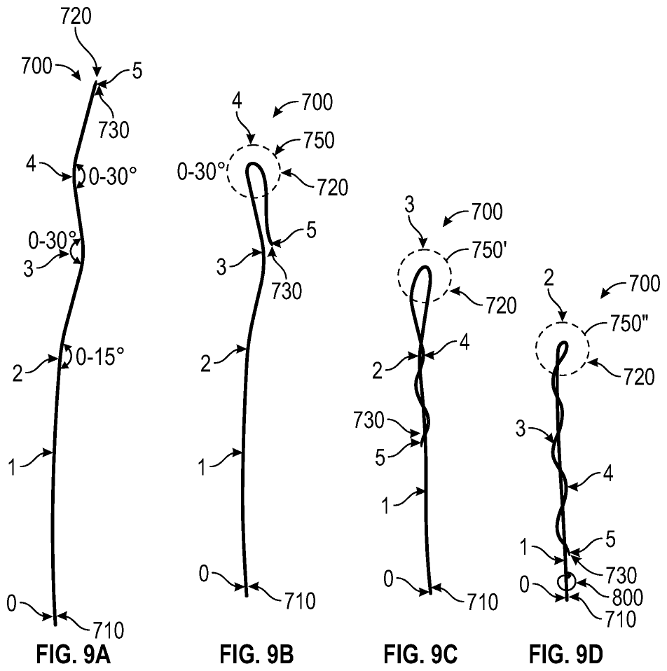

FIGS. 9A-9D illustrates a multi-variation looped wire for crossing a lesion according to some embodiments of the invention. In FIGS. 9A-9D, the looped wire 700 includes a first segment (0) that is the stiffest portion or stiff proximal end 710 of the looped wire 700. As further shown in FIGS. 9A-9D, the looped wire 700 further includes (i) a shaft (1) attached to the first segment (0), (ii) a second portion (2) having a first pre-set (or pre-shaped) angle that is between zero (0) and fifteen (15) degrees, (iii) a third portion (3) having a second pre-set (or pre-shaped) angle that is between zero (0) and thirty (30) degrees, (iv) a fourth portion (4) having a third pre-set (or pre-shaped) angle that is between zero (0) and thirty (30) degrees, and (v) a fifth portion (5) at the distal end 720 of the looped wire 700 that defines the end 730 of the looped wire 700. According to one embodiment, the highest stiffness of the looped wire 700 is located at the first segment (0), while the lowest stiffness of the looped wire 700 is located at the fifth portion (5) or end 730 of the looped wire 700. According to an embodiment, the first pre-set (or pre-shaped) angle at the second portion (2) of the looped wire 700 is structured to deliver the highest amount of force by the looped wire 700 (i.e., the highest amount of strength or gram force). The ability to deliver the highest amount of force at the first pre-set (or pre-shaped) angle at the second portion (2) of the looped wire 700 is due to the graduated stiffness of the looped wire 700.

As shown in FIG. 9A, the looped wire 700 includes a first configuration in which each of the segments or portions (0, 1, 2, 3, 4, 5) are disposed in a substantially straight line. FIG. 9B illustrates a second configuration in which the looped wire 700 is initially rotated by rotating the stiff, first segment (0) to create a loop 750 at the distal end 720 of the looped wire 700 (see, e.g., rotation 800 of the stiff, first segment (0) shown in FIG. 9D). As shown in the second configuration of FIG. 9B, the loop 750 is generated by the fourth portion (4) of the looped wire 700 having a third pre-set (or pre-shaped) angle that is between zero (0) and thirty (30) degrees. FIG. 9C illustrates a third configuration in which the looped wire 700 is further rotated by rotating the stiff, first segment (0) to create a loop 750′ at the distal end 720 of the looped wire 700 (see, e.g., rotation 800 of the stiff, first segment (0) shown in FIG. 9D). As shown in the third configuration of FIG. 9C, the loop 750′ is generated by the third portion (3) of the looped wire 700 having a second pre-set (or pre-shaped) angle that is between zero (0) and thirty (30) degrees. FIG. 9D illustrates a fourth configuration in which the looped wire 700 is even further rotated by rotating the stiff, first segment (0) to create a loop 750″ at the distal end 720 of the looped wire 700 (see, e.g., rotation 800 of the stiff, first segment (0) shown in FIG. 9D). As shown in the fourth configuration of FIG. 9D, the loop 750″ is generated by the second portion (2) of the looped wire 700 having a first pre-set (or pre-shaped) angle that is between zero (0) and fifteen (15) degrees. As further shown in the embodiments of FIGS. 9C and 9D, as the looped wire 700 is further rotated by rotating the stiff, first segment (0) to create a loop 750′ or 750″ at the distal end 720 of the looped wire 700, the fifth portion (5), the fourth portion (4), and/or the third portion (3) of the looped wire 700 begin to wrap around the shaft (1) of the looped wire 700.

According to an embodiment, a loop (see, e.g., loop 750 of FIG. 9B, loop 750′ of FIG. 9C, or loop 750″ of FIG. 9D) is generated when any segment (or portion) of the looped wire 700 bends (especially the portion or location of the looped wire 700 toward the tip or distal end 720 of the looped wire). Such a bend of a segment (or portion) of the looped wire 700 causes this segment to become parallel to a proximal segment (or portion) of the same looped wire (750), while generating a curve or loop at the distal end 720 of the looped wire 700 (see, e.g., loop 750 of FIG. 9B, loop 750′ of FIG. 9C, or loop 750″ of FIG. 9D). This type of curve or loop at the distal end 720 of the looped wire 700 (see, e.g., loop 750 of FIG. 9B, loop 750′ of FIG. 9C, or loop 750″ of FIG. 9D) carries a variable force due to the variation or graduation in the stiffness of the looped wire 700. For example, as shown in the embodiment of FIG. 9B, the fifth portion (5) has been bent by rotating the stiff, first segment (0) to create the loop 750. In this embodiment of FIG. 9B, when the fifth portion (5) is bent, this fifth portion (5) is now parallel to the third portion (3) of the looped wire 700 and the loop 750 is generated by the fourth portion (4) of the looped wire 700 having a third pre-set (or pre-shaped) angle that is between zero (0) and thirty (30) degrees. Both the third portion (3) of the looped wire 700 having a second pre-set (or pre-shaped) angle that is between zero (0) and thirty (30) degrees and the fourth portion (4) of the looped wire 700 having a third pre-set (or pre-shaped) angle that is between zero (0) and thirty (30) degrees help in generating a curve or loop at the distal end 720 of the looped wire 700 (see, e.g., loop 750 of FIG. 9B, loop 750′ of FIG. 9C, or loop 750″ of FIG. 9D). However, according to an embodiment, the curve or loop 750′ generated by the third portion (3) of the looped wire 700 (see, e.g., FIG. 9C) has (i) more force (i.e., strength or gram force) generated than the curve or loop 750 generated by the fourth portion (4) of the looped wire 700 (see, e.g., FIG. 9B) and (ii) less force (i.e., strength or gram force) generated than the curve or loop 750″ generated by the second portion (2) of the looped wire 700 (see, e.g., FIG. 9D). According to one embodiment, the curve or loop 750″ generated by the second portion (2) of the looped wire 700 having a first pre-set (or pre-shaped) angle that is between zero (0) and fifteen (15) degrees generates the highest force (i.e., strength or gram force), as compared to (i) the loop 750′ generated by the third portion (3) of the looped wire 700 or (ii) the loop 750 generated by the fourth portion (4) of the looped wire 700, due to the location of the second portion (2) on the stiffer portion of the shaft (1) of the looped wire 700. As discussed above, according to an embodiment, the shaft (1) of the looped wire 700 has a stiffness variation that varies between the first segment (0), which carries the highest stiffness of the looped wire 700, to the fifth portion (5) that defines the end 730 of the looped wire 700, which has the lowest stiffness of the looped wire 700.

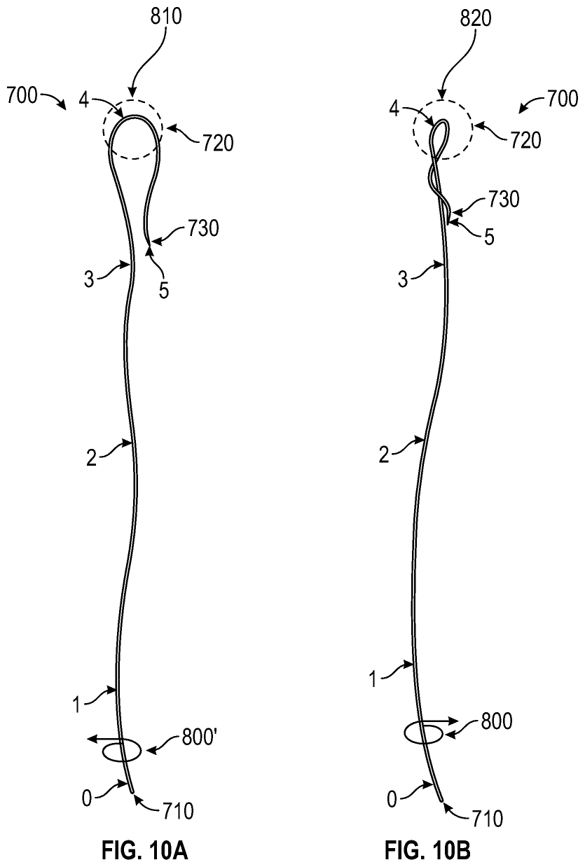

According to one embodiment, each loop (see, e.g., loop 750 of FIG. 9B, loop 750′ of FIG. 9C, or loop 750″ of FIG. 9D) generated by the various portions or angles of the looped wire 700 (e.g., the second portion (2) having a first pre-set (or pre-shaped) angle that is between zero (0) and fifteen (15) degrees, the third portion (3) having a second pre-set (or pre-shaped) angle that is between zero (0) and thirty (30) degrees, and/or the fourth portion (4) having a third pre-set (or pre-shaped) angle that is between zero (0) and thirty (30) degrees) has the ability to alter its force generation and force delivery by rotating the stiff, first segment (0) of the looped wire 700 (see, e.g., rotation 800 of the stiff, first segment (0) shown in FIG. 9D). For example, FIGS. 10A and 10B illustrate a further embodiment of the looped wire 700 of FIGS. 9A-9D, in which the rotation at the stiff, first segment (0) of the looped wire 700 generates either a loose loop 810 (as in the embodiment of FIG. 10A) or a tight loop 820 (as in the embodiment of FIG. 10B). As the looped wire 700 is rotated at the stiff, first segment (0) of the looped wire 700 (see, e.g., rotation 800 of FIG. 10B), torque is generated that ascends to the angled segment of the looped wire 700 (e.g., the second portion (2) having a first pre-set (or pre-shaped) angle that is between zero (0) and fifteen (15) degrees, the third portion (3) having a second pre-set (or pre-shaped) angle that is between zero (0) and thirty (30) degrees, and/or the fourth portion (4) having a third pre-set (or pre-shaped) angle that is between zero (0) and thirty (30) degrees). This torque, which is generated by the rotational motion of the looped wire 700, delivers energy that causes the loose loop 810 of FIG. 10A to tighten up and become the tight loop 820 of FIG. 10B, which is narrow in comparison to the original loose loop 810 of FIG. 10A. This tight loop 820 of FIG. 10B has increased gram weight or force that can be delivered to an occluded tissue in a vessel (e.g., a CTO and/or HGS). Since this force (e.g., strength or gram force) is variable, the force can be reduced by using a counter-spin 800′ (see, e.g., FIG. 10A). For example, as shown in the embodiment of FIG. 10A, the looped wire 700 can be rotated with a counter-rotation or spin 800′ at the stiff, first segment (0) of the looped wire 700 to cause the tight loop 820 of FIG. 10B to return to its original, baseline loose loop 810 of FIG. 10A. Thus, the torque generated by rotating the looped wire 700 at the stiff, first segment (0) of the looped wire 700 in the first direction (see, e.g., rotation 800 of FIG. 10B) causes the loop to tighten, while the rotating of the looped wire 700 at the stiff, first segment (0) of the looped wire 700 in the second or counter direction (see, e.g., counter-rotation or spin 800′ of FIG. 10A) causes the loop to loosen. This same method of increasing or decreasing the force of a loop via rotation (see, e.g., rotation 800 of FIG. 10) or counter-rotation (see, e.g., counter-rotation or spin 800′ of FIG. 10A) can be used on all of the loops discussed above (see, e.g., loop 750 of FIG. 9B, loop 750′ of FIG. 9C, or loop 750″ of FIG. 9D).

The structural formation of the wire can be accomplished by a variety of methods, for example, by forming the wire to have a looped shape during its original manufacture, or by applying heat and shaping forces to the wire after its initial formation. Once the wire has undergone structural formation, the wire maintains its structural formation when it is in a relaxed configuration, meaning that no forces are applied to it. When forces are applied to the wire that would change the configuration of the wire, the tensile forces in the wire resist the change. However, the wire may still flex and bend due to the applied forces.

The crossing wire can have varying stiffness and/or strength along its length. A particular stiffness and/or strength is chosen based on the application. Thus, according to some embodiments, a crossing wire is provided that has a predetermined pattern of variable strength (or stiffness) from a proximal end to a distal end of the crossing wire. The crossing wire can include markers that indicate the proper position of the wire for a particular stiffness and/or strength. Occlusions providing mild resistance can be crossed with a less stiff or strong portion of the wire, while severe occlusions can be crossed with the stiffest or strongest portion of the wire. According to one configuration, the crossing wire has three different stiffness values and/or strength values along its length.

According to some aspects of the invention, the wire can be adapted for use in all arteries and veins. The gram tip stiffness of the wire can start at 1-3 grams. The wire can be made from a hydrophilic or non-hydrophilic material, and the choice of the material may be based on the lesion. The crossing wire can be encased in an outer shell. The outer shell can prevent the proximal end of the secondary shaft from inadvertently catching on tissue. The outer shell may be useful when navigating the crossing wire through particular veins and arteries, for example, the aortic junction.

The force generation and stiffness of the crossing wire can be based on a mechanical configuration change, and hence the stiffness can be variable. However, the wire can also have a configuration in which the distal end of the wire applies a specific force that is constant. For example, the crossing wire can be formed to have a closed loop, meaning that the primary and secondary shafts are bonded or welded such that the loop has a predetermined stiffness.

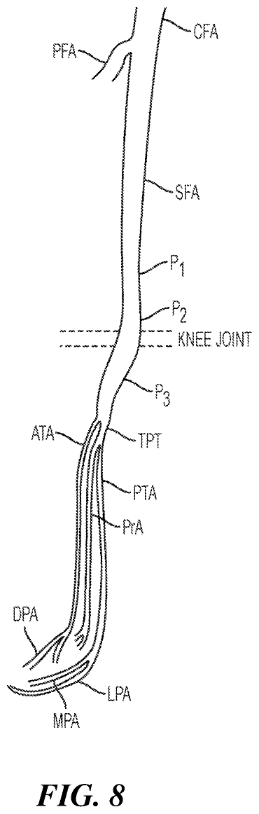

Existing CTO crossing devices are too large to be used in the arteries below the knee. The present device can have a size that allows it to be used below the knee, for example, throughout the vasculature illustrated in FIG. 8. The device can be used in vessels having a diameter between 1.5 mm and 30 mm, according to some aspects. According to one aspect, the catheter is a 0.035″ catheter. According to one aspect, the crossing wire is a 0.018″ wire. The embodiments of the invention are not limited to these dimensions.

The CTO specialty wire can have the same or varying degrees and/or combinations of rigidity and/or column strength so that the loop at the end can be moved in and out to the desired portion/rigidity/strength wire for a particular application. The combination(s) of rigidity can be predetermined.

According to some embodiments, the crossing wire is formed as a singular or unitary wire with each of the wire segments (see, e.g., wire segments 6, 7, 8, 9, 10, 11 of FIG. 3) and each of the links (see, e.g., links 1, 2, 3, 4, 5 of FIG. 3) formed therein. According to an embodiment, each of the wire segments (see, e.g., wire segments 6, 7, 8, 9, 11 of FIG. 3) and each of the links (see, e.g., links 1, 2, 3, 4, 5 of FIG. 3) comprise independent, separate pieces that are attached or connected together to make the crossing wire.

According to some embodiments, each of the wire segments (see, e.g., wire segments 6, 7, 8, 9, 10, 11 of FIG. 3) and each of the links (see, e.g., links 1, 2, 3, 4, 5 of FIG. 3) that alternate with the plurality of wire segments (see, e.g., wire segments 6, 7, 8, 9, 11 of FIG. 3) comprise nitinol of a certain flexibility, strength, and/or diameter.

According to some embodiments, the above-described crossing wires provide flexibility when needed and stiffness or strength when needed through the variation in gram force or stiffness of each of the links (see, e.g., links 1, 2, 3, 4, 5 of FIG. 3) and/or each of the wire segments (see, e.g., wire segments 6, 7, 8, 9, 10, 11 of FIG. 3) of the crossing wire from the distal end to the proximal end of the crossing wire. Such variation in flexibility and/or stiffness or strength can allow for less crossing wires needed for interrogating certain lesions (e.g., CTO and/or HGS), such that the amount of wire exchanges can be reduced, including by, e.g., up to fifty percent.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

Further aspects of the present disclosure are provided by the subject matter of the following clauses.

A device for crossing a lesion in a tissue lumen, with the device comprising a crossing wire configured to pass through a lumen of a catheter, the crossing wire comprising a plurality of wire segments and the crossing wire is configured to form a loop at a distal end of the crossing wire.

The device for crossing a lesion according to any preceding clause, wherein the crossing wire includes the loop.

The device for crossing a lesion according to any preceding clause, wherein the loop comprises at least two wire segments with a link between the at least two wire segments.

The device for crossing a lesion according to any preceding clause, wherein the link has a variable gram force based on a wire diameter of the link.

The device for crossing a lesion according to any preceding clause, wherein the link has at least three variations in gram force based on the wire diameter of the link.

The device for crossing a lesion according to any preceding clause, wherein the link generates a gram force from a combination of the at least two wire segments that the link is positioned between.

The device for crossing a lesion according to any preceding clause, wherein the link comes into contact with the lesion.

The device for crossing a lesion according to any preceding clause, wherein the plurality of wire segments includes a plurality of wire links, with each wire link having a variable strength.

The device for crossing a lesion according to any preceding clause, wherein each wire link of the plurality of wire links that has a variable strength alternates with a wire segment having a constant strength.

The device for crossing a lesion according to any preceding clause, wherein a wire link of the plurality of wire links is positioned at the distal end of the crossing wire.

The device for crossing a lesion according to any preceding clause, wherein the wire link positioned at the distal end of the crossing wire has the lowest tensile strength.

The device for crossing a lesion according to any preceding clause, wherein the wire link positioned at the distal end of the crossing wire has a tensile strength of less than 1 gram.

The device for crossing a lesion according to any preceding clause, wherein a wire link of the plurality of wire links is positioned at a proximal end of the crossing wire.

The device for crossing a lesion according to any preceding clause, wherein the wire link positioned at the proximal end of the crossing wire has the highest tensile strength.

The device for crossing a lesion according to any preceding clause, wherein the wire link positioned at the proximal end of the crossing wire has a tensile strength of greater than 200 grams.

The device for crossing a lesion according to any preceding clause, wherein each wire link of the plurality of wire links increases in stiffness (or strength) from the distal end to a proximal end of the crossing wire.

The device for crossing a lesion according to any preceding clause, wherein each wire link of the plurality of wire links has a strength of less than 1 gram to about 200 grams.

The device for crossing a lesion according to any preceding clause, wherein each wire link of the plurality of wire links increases in diameter from the distal end to a proximal end of the crossing wire.

The device for crossing a lesion according to any preceding clause, wherein the diameter of each wire link of the plurality of wire links is one of (i) 0.014 inches, (ii) 0.018 inches, or (iii) 0.035 inches.

The device for crossing a lesion according to any preceding clause, wherein the plurality of wire segments comprises at least (i) a first segment at a proximal end of the crossing wire having a stiffness that is the highest stiffness of the crossing wire and (ii) a shaft attached to the first segment.

The device for crossing a lesion according to any preceding clause, wherein the plurality of wire segments further comprises one or more of (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees.

The device for crossing a lesion according to any preceding clause, wherein the plurality of wire segments further comprises a second segment at the distal end of the crossing wire having a stiffness that is at least one of (i) less than the stiffness of the first segment, or (ii) the lowest stiffness of the crossing wire.

The device for crossing a lesion according to any preceding clause, wherein the loop of the crossing wire is configured to be rotated (i) clockwise at 180 degrees and/or (ii) counterclockwise at 180 degrees.

The device for crossing a lesion according to any preceding clause, wherein rotation of the loop narrows the size of the loop, which results in a loop that is straight with a smaller diameter and a higher strength.

The device for crossing a lesion according to any preceding clause, wherein the loop of the crossing wire is configured to be rotated (i) in a first direction and (ii) a second direction that is opposite to the first direction.

The device for crossing a lesion according to any preceding clause, wherein rotation of the loop in (i) the first direction narrows the size of the loop, which results in a loop having a higher strength, and (ii) the second direction increases the size of the loop, which results in the loop having a lower strength.

The device for crossing a lesion according to any preceding clause, wherein the lesion comprises one or more of a chronic total occlusion (CTO) or a high grade stenosis (HGS).

The device for crossing a lesion according to any preceding clause, wherein a proximal end of the crossing wire has a first stiffness, and the loop has a second stiffness, wherein the first stiffness is greater than the second stiffness.

The device for crossing a lesion according to any preceding clause, wherein the crossing wire comprises at least two secondary wires that are twisted together to form the crossing wire.

The device for crossing a lesion according to any preceding clause, wherein the crossing wire comprises at least three secondary wires that are twisted together to form the crossing wire.

The device for crossing a lesion according to any preceding clause, wherein the crossing wire is configured to be rotatable back and forth through an angle less than 360 degrees while maintaining contact with the lesion to erode the lesion.

The device for crossing a lesion according to any preceding clause, wherein the crossing wire is configured to be rotatable back and forth through an angle of about 180 degrees while maintaining contact with the lesion to erode the lesion.

The device for crossing a lesion according to any preceding clause, wherein the crossing wire has a variable stiffness along its length.

The device for crossing a lesion according to any preceding clause, wherein the loop includes a material that is radiopaque.

A device for crossing a lesion in a tissue lumen, with the device comprising a catheter and the crossing wire according to any preceding clause.

A method for crossing a chronic total occlusion (CTO) and/or a high grade stenosis (HGS), the method comprising inserting a catheter having a crossing wire disposed in a lumen of the catheter into an occluded vessel, the crossing wire comprising a plurality of wire segments and a loop at a distal end of the crossing wire; extending the loop of the crossing wire beyond a distal end of the catheter to contact an occlusion; grasping the crossing wire at a position proximal to a proximal end of the catheter; and rotating the grasped crossing wire back and forth through an angle less than 360 degrees while maintaining the loop of the crossing wire in contact with the occlusion to erode the occlusion.

The method according to any preceding clause, further comprising twisting the grasped crossing wire through an angle of about 180 degrees while pressing the loop of the crossing wire against the occlusion.

The method according to any preceding clause, wherein the plurality of wire segments comprises at least (i) a first segment at the proximal end of the crossing wire having a stiffness that is the highest stiffness of the crossing wire and (ii) a shaft attached to the first segment.

The method according to any preceding clause, wherein the crossing wire is grasped at the first segment at the proximal end of the crossing wire and the first segment is rotated.

The method according to any preceding clause, wherein the plurality of wire segments further comprises (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees.

The method according to any preceding clause, wherein the step of twisting the grasped crossing wire causes the loop to form at one of the second portion, the third portion, or the fourth portion.

The method according to any preceding clause, wherein the plurality of wire segments further comprises a second segment at the distal end of the crossing wire having a stiffness that is at least one of (i) less than the stiffness of the first segment, or (ii) the lowest stiffness of the crossing wire.

The method according to any preceding clause, further comprising at least one of (a) rotating the grasped crossing wire in a first direction, or (b) rotating the grasped crossing wire in a second direction that is opposite to the first direction, wherein rotating the grasped crossing wire in the first direction narrows the size of the loop, which results in a loop having a higher strength, and wherein rotating the grasped crossing wire in the second direction increases the size of the loop, which results in the loop having a lower strength.

A device for crossing a lesion in a tissue lumen, the device comprising a crossing wire having a predetermined pattern of variable strength from a proximal end to a distal end of the crossing wire.

The device for crossing a lesion according to any preceding clause, wherein the crossing wire is configured to form a loop at the distal end of the crossing wire.

The device for crossing a lesion according to any preceding clause, wherein the crossing wire further comprises a loop formed at the distal end of the crossing wire.

The device for crossing a lesion according to any preceding clause, wherein the crossing wire comprises a plurality of wire segments.

The device for crossing a lesion according to any preceding clause, wherein the plurality of wire segments includes a plurality of wire links, with each wire link having a variable strength.

The device for crossing a lesion according to any preceding clause, wherein each wire link of the plurality of wire links that has a variable strength alternates with a wire segment having a constant strength.

The device for crossing a lesion according to any preceding clause, wherein a wire link of the plurality of wire links is positioned at the distal end of the crossing wire.

The device for crossing a lesion according to any preceding clause, wherein the wire link positioned at the distal end of the crossing wire has the lowest tensile strength.

The device for crossing a lesion according to any preceding clause, wherein the wire link positioned at the distal end of the crossing wire has a tensile strength of less than 1 gram.

The device for crossing a lesion according to any preceding clause, wherein a wire link of the plurality of wire links is positioned at the proximal end of the crossing wire.

The device for crossing a lesion according to any preceding clause, wherein the wire link positioned at the proximal end of the crossing wire has the highest tensile strength.

The device for crossing a lesion according to any preceding clause, wherein the wire link positioned at the proximal end of the crossing wire has a tensile strength of greater than 200 grams.

The device for crossing a lesion according to any preceding clause, wherein each wire link of the plurality of wire links increases in stiffness (or strength) from the distal end to a proximal end of the crossing wire.

The device for crossing a lesion according to any preceding clause, wherein each wire link of the plurality of wire links has a strength of less than 1 gram to about 200 grams.

The device for crossing a lesion according to any preceding clause, wherein each wire link of the plurality of wire links increases in diameter from the distal end to a proximal end of the crossing wire.

The device for crossing a lesion according to any preceding clause, wherein the diameter of each wire link of the plurality of wire links is one of (i) 0.014 inches, (ii) 0.018 inches, or (iii) 0.035 inches.

The device for crossing a lesion according to any preceding clause, wherein the plurality of wire segments comprises at least (i) a first segment at a proximal end of the crossing wire having a stiffness that is the highest stiffness of the crossing wire and (ii) a shaft attached to the first segment.

The device for crossing a lesion according to any preceding clause, wherein the plurality of wire segments further comprises one or more of (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees.

The device for crossing a lesion according to any preceding clause, wherein the plurality of wire segments further comprises a second segment at the distal end of the crossing wire having a stiffness that is at least one of (i) less than the stiffness of the first segment, or (ii) the lowest stiffness of the crossing wire.

The device for crossing a lesion according to any preceding clause, wherein the lesion comprises one or more of a chronic total occlusion (CTO) or a high grade stenosis (HGS).

The device for crossing a lesion according to any preceding clause, wherein the proximal end of the crossing wire has a first stiffness, and the loop has a second stiffness, wherein the first stiffness is greater than the second stiffness.

I claim:

1. A device for crossing a lesion in a tissue lumen, the device comprising:
a crossing wire configured to pass through a lumen of a catheter, the crossing wire comprising a plurality of wire segments and the crossing wire is configured to form a loop at a distal end of the crossing wire.

2. The device for crossing a lesion according to claim 1, wherein the crossing wire includes the loop.

3. The device for crossing a lesion according to claim 1, wherein the loop comprises at least two wire segments with a link between the at least two wire segments.

4. The device for crossing a lesion according to claim 3, wherein the link has a variable gram force based on a wire diameter of the link.

5. The device for crossing a lesion according to claim 4, wherein the link has at least three variations in gram force based on the wire diameter of the link.

6. The device for crossing a lesion according to claim 3, wherein the link generates a gram force from a combination of the at least two wire segments that the link is positioned between.

7. The device for crossing a lesion according to claim 3, wherein the link comes into contact with the lesion.

8. The device for crossing a lesion according to claim 1, wherein the plurality of wire segments includes a plurality of wire links, with each wire link having a variable strength.

9. The device for crossing a lesion according to claim 8, wherein each wire link of the plurality of wire links that has a variable strength alternates with a wire segment having a constant strength.

10. The device for crossing a lesion according to claim 8, wherein a wire link of the plurality of wire links is positioned at the distal end of the crossing wire.

11. The device for crossing a lesion according to claim 10, wherein the wire link positioned at the distal end of the crossing wire has the lowest tensile strength.

12. The device for crossing a lesion according to claim 10, wherein the wire link positioned at the distal end of the crossing wire has a tensile strength of less than 1 gram.

13. The device for crossing a lesion according to claim 8, wherein a wire link of the plurality of wire links is positioned at a proximal end of the crossing wire.

14. The device for crossing a lesion according to claim 13, wherein the wire link positioned at the proximal end of the crossing wire has the highest tensile strength.

15. The device for crossing a lesion according to claim 13, wherein the wire link positioned at the proximal end of the crossing wire has a tensile strength of greater than 200 grams.

16. The device for crossing a lesion according to claim 8, wherein each wire link of the plurality of wire links increases in stiffness (or strength) from the distal end to a proximal end of the crossing wire.

17. The device for crossing a lesion according to claim 8, wherein each wire link of the plurality of wire links has a strength of less than 1 gram to about 200 grams.

18. The device for crossing a lesion according to claim 8, wherein each wire link of the plurality of wire links increases in diameter from the distal end to a proximal end of the crossing wire.

19. The device for crossing a lesion according to claim 18, wherein the diameter of each wire link of the plurality of wire links is one of (i) 0.014 inches, (ii) 0.018 inches, or (iii) 0.035 inches.

20. The device for crossing a lesion according to claim 1, wherein the plurality of wire segments comprises at least (i) a first segment at a proximal end of the crossing wire having a stiffness that is the highest stiffness of the crossing wire and (ii) a shaft attached to the first segment.

21. The device for crossing a lesion according to claim 20, wherein the plurality of wire segments further comprises one or more of (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees.

22. The device for crossing a lesion according to claim 20, wherein the plurality of wire segments further comprises a second segment at the distal end of the crossing wire having a stiffness that is at least one of (i) less than the stiffness of the first segment, or (ii) the lowest stiffness of the crossing wire.

23. The device for crossing a lesion according to claim 1, wherein the loop of the crossing wire is configured to be rotated (i) clockwise at 180 degrees and/or (ii) counterclockwise at 180 degrees.

24. The device for crossing a lesion according to claim 23, wherein rotation of the loop narrows the size of the loop, which results in a loop that is straight with a smaller diameter and a higher strength.

25. The device for crossing a lesion according to claim 1, wherein the loop of the crossing wire is configured to be rotated (i) in a first direction and (ii) a second direction that is opposite to the first direction.

26. The device for crossing a lesion according to claim 25, wherein rotation of the loop in (i) the first direction narrows the size of the loop, which results in a loop having a higher strength, and (ii) the second direction increases the size of the loop, which results in the loop having a lower strength.

27. The device for crossing a lesion according to claim 1, wherein the lesion comprises one or more of a chronic total occlusion (CTO) or a high grade stenosis (HGS).

28. The device for crossing a lesion according to claim 1, wherein a proximal end of the crossing wire has a first stiffness, and the loop has a second stiffness, wherein the first stiffness is greater than the second stiffness.

29. The device for crossing a lesion according to claim 1, wherein the crossing wire comprises at least two secondary wires that are twisted together to form the crossing wire.

30. The device for crossing a lesion according to claim 1, wherein the crossing wire comprises at least three secondary wires that are twisted together to form the crossing wire.

31. The device for crossing a lesion according to claim 1, wherein the crossing wire is configured to be rotatable back and forth through an angle less than 360 degrees while maintaining contact with the lesion to erode the lesion.

32. The device for crossing a lesion according to claim 1, wherein the crossing wire is configured to be rotatable back and forth through an angle of about 180 degrees while maintaining contact with the lesion to erode the lesion.

33. The device for crossing a lesion according to claim 1, wherein the crossing wire has a variable stiffness along its length.

34. The device for crossing a lesion according to claim 1, wherein the loop includes a material that is radiopaque.

35. A device for crossing a lesion in a tissue lumen, comprising:
a catheter; and
the crossing wire according to claim 1.

36. A method for crossing a chronic total occlusion (CTO) and/or a high grade stenosis (HGS), the method comprising:
inserting a catheter having a crossing wire disposed in a lumen of the catheter into an occluded vessel, the crossing wire comprising a plurality of wire segments and a loop at a distal end of the crossing wire;
extending the loop of the crossing wire beyond a distal end of the catheter to contact an occlusion;
grasping the crossing wire at a position proximal to a proximal end of the catheter; and
rotating the grasped crossing wire back and forth through an angle less than 360 degrees while maintaining the loop of the crossing wire in contact with the occlusion to erode the occlusion.

37. The method according to claim 36, further comprising:
twisting the grasped crossing wire through an angle of about 180 degrees while pressing the loop of the crossing wire against the occlusion.

38. The method according to claim 37, wherein the plurality of wire segments comprises at least (i) a first segment at the proximal end of the crossing wire having a stiffness that is the highest stiffness of the crossing wire and (ii) a shaft attached to the first segment.

39. The method according to claim 38, wherein the crossing wire is grasped at the first segment at the proximal end of the crossing wire and the first segment is rotated.

40. The method according to claim 38, wherein the plurality of wire segments further comprises (i) a second portion having a first pre-set angle that is between zero degrees and fifteen degrees, (iii) a third portion having a second pre-set angle that is between zero degrees and thirty degrees, and (iii) a fourth portion having a third pre-set angle that is between zero degrees and thirty degrees.

41. The method according to claim 40, wherein the step of twisting the grasped crossing wire causes the loop to form at one of the second portion, the third portion, or the fourth portion.

42. The method according to claim 38, wherein the plurality of wire segments further comprises a second segment at the distal end of the crossing wire having a stiffness that is at least one of (i) less than the stiffness of the first segment, or (ii) the lowest stiffness of the crossing wire.

43. The method according to claim 36, further comprising at least one of:
(a) rotating the grasped crossing wire in a first direction, or
(b) rotating the grasped crossing wire in a second direction that is opposite to the first direction,
wherein rotating the grasped crossing wire in the first direction narrows the size of the loop, which results in a loop having a higher strength, and
wherein rotating the grasped crossing wire in the second direction increases the size of the loop, which results in the loop having a lower strength.
